(19) United States
(12) Patent Application Publication
(10) Pub. No.: US 2026/0191754 A1
(43) Pub. Date: Jul. 9, 2026

(54) PROCESS FOR TREATING KERATIN FIBRES USING A COMPOSITION COMPRISING AT LEAST TWO DIFFERENT PARTICULAR FATTY ACIDS, THE COMPOSITION HAVING A LOW SILICONE CONTENT

(71) Applicant: L'OREAL, Paris (FR)

(72) Inventors: Fatima MAKHLOUF, Aulnay-sous-Bois (FR); Stéphane SABELLE, Aulnay-sous-Bois (FR)

(73) Assignee: L'OREAL, Paris (FR)

(21) Appl. No.: 19/135,638

(22) PCT Filed: Dec. 14, 2023
(86) PCT No.: PCT/EP2023/085965

§ 371 Date: Jun. 4, 2025

(30) Foreign Application Priority Data
Dec. 16, 2022 (FR) FR2213688

(51) Int. Cl. A61K 8/41; A61K 8/37; A61Q 5/06; A61Q 5/12

(52) U.S. Cl. CPC A61K 8/416; A61K 8/37; A61Q 5/06; A61Q 5/12

(57) ABSTRACT

The present invention relates to a process for treating keratin fibres using a composition comprising two different particular fatty acids, the composition having a low silicone content.

### RELATED APPLICATIONS

The present application is a national stage application that claims priority to International Application No. PCT/EP2023/085965 filed Dec. 14, 2023, which claims priority to French Application No. 2213688 filed Dec. 16, 2022, the disclosures of which are incorporated herein by reference in their entirety.

### TECHNICAL FIELD OF THE INVENTION

The present invention relates to a process for treating keratin fibres using a composition comprising at least two different particular fatty acids, the composition having a low silicone content.

### BACKGROUND OF THE INVENTION

The hair is damaged and weakened by external atmospheric agents such as pollution and bad weather, and also by mechanical or chemical treatments, such as brushing, combing, dyeing, bleaching, permanent-waving, relaxing and repeated washing. The hair thus becomes damaged and may in the long run become dry, coarse, brittle, dull, split and/or soft or else sensitive to humidity, making the hair unmanageable, often with frizziness, and/or difficult to style in a humid environment, in particular in a very humid environment.

Thus, to overcome these drawbacks, it is common practice to resort to hair treatments which make use of compositions intended for conditioning the hair appropriately by giving it satisfactory cosmetic properties, notably a soft feel (the hair is no longer coarse), good disentangling properties leading to easy combing, and good manageability of the hair which is thus easy to shape.

However, these hair treatments, which are often based on silicone-based starting materials, may give the hair a greasy, heavy feel that is not always pleasant for the user. Moreover, these hair treatments have little or no effect on controlling the volume of keratin fibres and/or maintaining the hairstyle and/or hair discipline in a humid or very humid environment.

There is thus a real need to develop a hair treatment process that can remedy these drawbacks and that is notably capable of preserving or even improving the quality of the fibre, notably the softness, disentangling, smoothness, discipline and/or sheen, and of durably limiting the formation of frizziness and/or the increase in volume of the head of hair in a humid environment so as to facilitate styling of the hair in a humid environment. Such a process should ideally not use silicone-based starting materials.

The Applicant has discovered, surprisingly, that all or some of these objectives can be achieved by the process according to the present invention.

### SUMMARY OF THE INVENTION

According to a first aspect, a subject of the present invention is a process for treating keratin fibres, comprising step i) of applying to the keratin fibres a composition (C) comprising at least two different compounds D chosen independently from the compounds of formula (I) below, the salts thereof, the isomers thereof, and the solvates thereof, such as hydrates, and mixtures thereof:

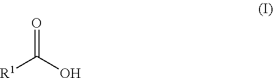

in which formula (I):
R¹ represents:
a linear or branched, saturated or unsaturated hydrocarbon-based group comprising from 6 to 26 carbon atoms, the hydrocarbon-based group being optionally substituted with one or more identical or different groups chosen from: hydroxyl (—OH), amino (—NH₂), carboxyl (—COOH), (hetero)cyclic such as phenyl and/or optionally interrupted with one or more heteroatoms or groups chosen from —O—, —CO—, —NR^{a}—, or combinations thereof such as —O—CO—, —(CO)—O—, —NR^{a}—(CO)— or —(CO)—NR^{a}—; or
a (hetero)cyclic group such as phenyl, optionally substituted with one or more identical or different groups chosen from: hydroxyl (—OH) or R^{b}—(CO)—;
R^{a} represents a hydrogen atom or a linear or branched, saturated or unsaturated hydrocarbon-based group comprising from 1 to 10 carbon atoms optionally substituted with one or more identical or different groups chosen from (—OH), amino (—NH₂) or carboxyl (—COOH);
R^{b} represents a (C₂-C₁₀)alkyl group;
   it being understood that:
the salts of the compounds of formula (I) are chosen from monovalent salts of the compounds of formula (I), preferably chosen from alkali metal salts such as sodium or potassium salts, ammonium salts, tetra(hydroxy)(C₁-C₁₀)alkylammonium salts, di(hydroxy)(C₁-C₁₀)alkylammonium salts, tri(hydroxy)(C₁-C₁₀)alkylammonium salts, and mixtures thereof, more preferentially chosen from alkali metal salts such as sodium or potassium salts, tetra(hydroxy)(C₁-C₁₀)alkylammonium salts, and mixtures thereof; and
composition (C) comprises a total silicone content of less than 5% by weight relative to the total weight of composition (C).

According to a second aspect, a subject of the present invention is a composition (C) as defined previously.

According to a third aspect, a subject of the present invention is the use of a composition (C) as defined previously for caring for keratin fibres, preferably for giving keratin fibres a soft and/or smooth feel and/or sheen, more preferentially for giving them a soft and/or smooth feel.

According to a fourth aspect, a subject of the present invention is the use of a composition (C) as defined previously for protecting keratin fibres from moisture, preferably for limiting the formation of frizziness and/or the increase in volume of the head of hair in a humid environment.

### DETAILED DESCRIPTION OF THE INVENTION

For the purposes of the present invention and unless otherwise indicated:
the term “keratin fibres” means fibres of human or animal origin, such as head hair, bodily hairs, the eyelashes, the eyebrows, wool, angora, cashmere or fur. According to the present invention, the keratin fibres are preferably human keratin fibres, more preferentially the head hair.
the term “alkyl group” means a saturated, linear or branched hydrocarbon-based radical comprising from 1 to 30 carbon atoms, preferentially from 1 to 26 carbon atoms, more preferentially from 1 to 22 carbon atoms, for example methyl, ethyl, n-propyl, isopropyl, butyl, n-pentyl, n-hexyl, n-decyl, n-undecyl, n-dodecyl, n-tetradecyl, n-hexadecyl or eicosyl.
the term “(Cₓ-C_{y})alkyl group” means an alkyl group comprising from x to y carbon atoms.
the term “hydroxy(Cₓ-C_{y})alkyl group” means a (Cₓ-C_{y})alkyl group, at least one of the hydrogen atoms of which is optionally replaced with a hydroxyl (—OH) group. A (hydroxy)(Cₓ-C_{y})alkyl group thus denotes a (Cₓ-C_{y})alkyl group or a hydroxyl(Cₓ-C_{y})alkyl group, i.e. a (Cₓ-C_{y})alkyl group in which at least one of the hydrogen atoms is replaced with a hydroxyl (—OH) group.
the term “di(hydroxy)(Cₓ-C_{y})alkylammonium salt” means an ammonium salt bearing two (hydroxyl)(Cₓ-C_{y})alkyl groups, said (hydroxyl)(Cₓ-C_{y})alkyl groups being identical or different.
the term “tri(hydroxy)(Cₓ-C_{y})alkylammonium salt” means an ammonium salt bearing three (hydroxyl)(Cₓ-C_{y})alkyl groups, said (hydroxyl)(Cₓ-C_{y})alkyl groups being identical or different.
the term “tetra(hydroxy)(Cₓ-C_{y})alkylammonium salt” means an ammonium salt bearing four (hydroxyl)(Cₓ-C_{y})alkyl groups, said (hydroxyl)(Cₓ-C_{y})alkyl groups being identical or different.
the term “(hetero)cyclic group” means a cyclic or heterocyclic group.
the term “cyclic group” means a monocyclic or condensed or non-condensed, saturated or unsaturated, notably aromatic, polycyclic carbocycle comprising from 6 to 22 carbon atoms, it being possible for said cyclic group to be substituted with one or more identical or different groups, notably chosen from: (C₁-C₆)alkyl, (C₁-C₆)alkoxy, (C₁-C₆)(poly)hydroxyalkyl, hydroxyl (—OH) or carboxyl (—COOH).
the term “heterocyclic group” means a monocyclic or condensed or non-condensed, saturated or unsaturated, in particular aromatic, polycyclic group comprising from 5 to 22 members and containing from 1 to 3 heteroatoms chosen from a nitrogen, oxygen or sulfur atom, it being possible for said heterocyclic group to be substituted with one or more identical or different groups, notably chosen from: (C₁-C₆)alkyl, (C₁-C₆)alkoxy, (C₁-C₆)(poly)hydroxyalkyl, hydroxyl (—OH) or carboxyl (—COOH).
the term “unsaturated hydrocarbon-based group” means a hydrocarbon-based group comprising one or more conjugated or non-conjugated ethylenic double bonds.
the term “isomer” means an optical, geometrical or tautomeric isomer.

The terms “at least one” and “one or more” are synonymous and may be used interchangeably.

### Process for Treating Keratin Fibres

According to a first aspect, a subject of the present invention is a process for treating keratin fibres as defined previously.

The Applicant has found, surprisingly, that the process according to the present invention made it possible to maintain or even improve the quality of the fibre, notably the softness, disentangling, smoothness and/or discipline and/or sheen, and to limit the formation of frizziness and/or the increase in volume of the head of hair in a humid environment, thus making it easier to style the hair in a humid environment.

According to a preferred embodiment, the salts of the compounds of formula (I) are chosen from salts of alkali metals such as sodium or potassium, ammonium salts and tetra(C₁-C₁₀)alkylammonium salts, di(C₁-C₁₀)alkylammonium salts, tri(C₁-C₁₀)alkylammonium salts, and mixtures thereof, preferably chosen from salts of alkali metals such as sodium or potassium, tetra(C₁-C₁₀)alkylammonium salts, and mixtures thereof.

According to a preferred embodiment, R¹ represents a linear or branched, saturated or unsaturated hydrocarbon-based group comprising from 6 to 26 carbon atoms, the hydrocarbon-based group being optionally substituted with one or more identical or different groups chosen from: hydroxyl (—OH), amino (—NH₂), carboxyl (—COOH), (hetero)cyclic such as phenyl and/or optionally interrupted with one or more heteroatoms or groups chosen from —O—, —CO—, —NR^{a}—, or combinations thereof such as —O—CO—, —(CO)—O—, —NR^{a}—(CO)— or —(CO)—NR^{a}—, preferably an unsubstituted, saturated or unsaturated, linear or branched hydrocarbon-based group comprising from 6 to 26 carbon atoms, more preferentially an unsubstituted, saturated or unsaturated, linear or branched hydrocarbon-based group comprising from 6 to 20 carbon atoms.

According to one embodiment, composition (C) does not comprise any salts of the compounds of formula (I) as defined previously.

According to a preferred embodiment, at least one of the different compounds D, preferably all of the different compounds D, are in non-salified form.

According to a preferred embodiment, at least two of the different compounds D are in non-salified form and the mole ratio of the total amount of one of the different compounds D in non-salified form to the total amount of another of the different compounds D in non-salified form in composition (C) ranges from 0.25 to 4.

In cases where composition (C) comprises more than two different compounds D in non-salified form, it is sufficient for the mole ratio between two of these different compounds D to vary from 0.25 to 4 for this criterion to be met.

According to a particular embodiment, at least one of the different compounds D is in non-salified form and at least one of the different compounds D is in salified form and the mole ratio of the total amount of one of the different compounds D in non-salified form to the total amount of one of the different compounds D in salified form in composition (C) is greater than or equal to 1, and preferably ranges from 1 to 100.

In cases where composition (C) comprises more than two different compounds D in non-salified form and/or more than two different compounds D in salified form, it is sufficient for the mole ratio of the total amount of one of the different compounds D in non-salified form to the total amount of one of the different compounds D in salified form to be greater than or equal to 1, preferably ranging from 1 to 100, in order for this criterion to be met.

Composition (C) preferably comprises not more than five different compounds D, more preferentially not more than four different compounds D, even more preferentially not more than three different compounds D, and better still not more than two different compounds D, chosen independently from the compounds of formula (I) as defined previously, the salts thereof, the isomers thereof, and the solvates thereof, such as hydrates, and mixtures thereof.

According to a preferred embodiment, the different compounds D are chosen from the following compounds 1 to 39, the salts thereof, the isomers thereof, and the solvates thereof, such as hydrates, and mixtures thereof:

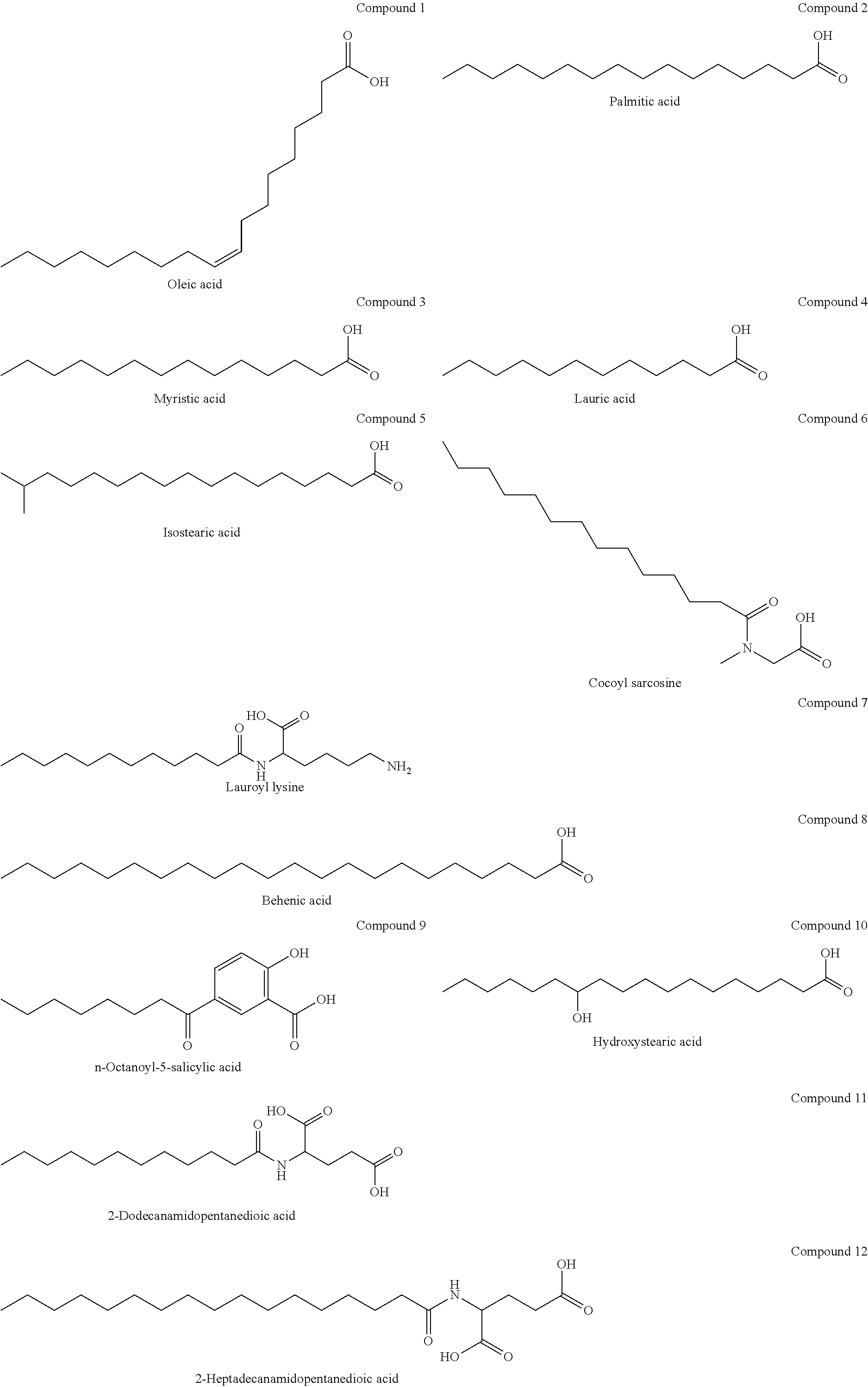

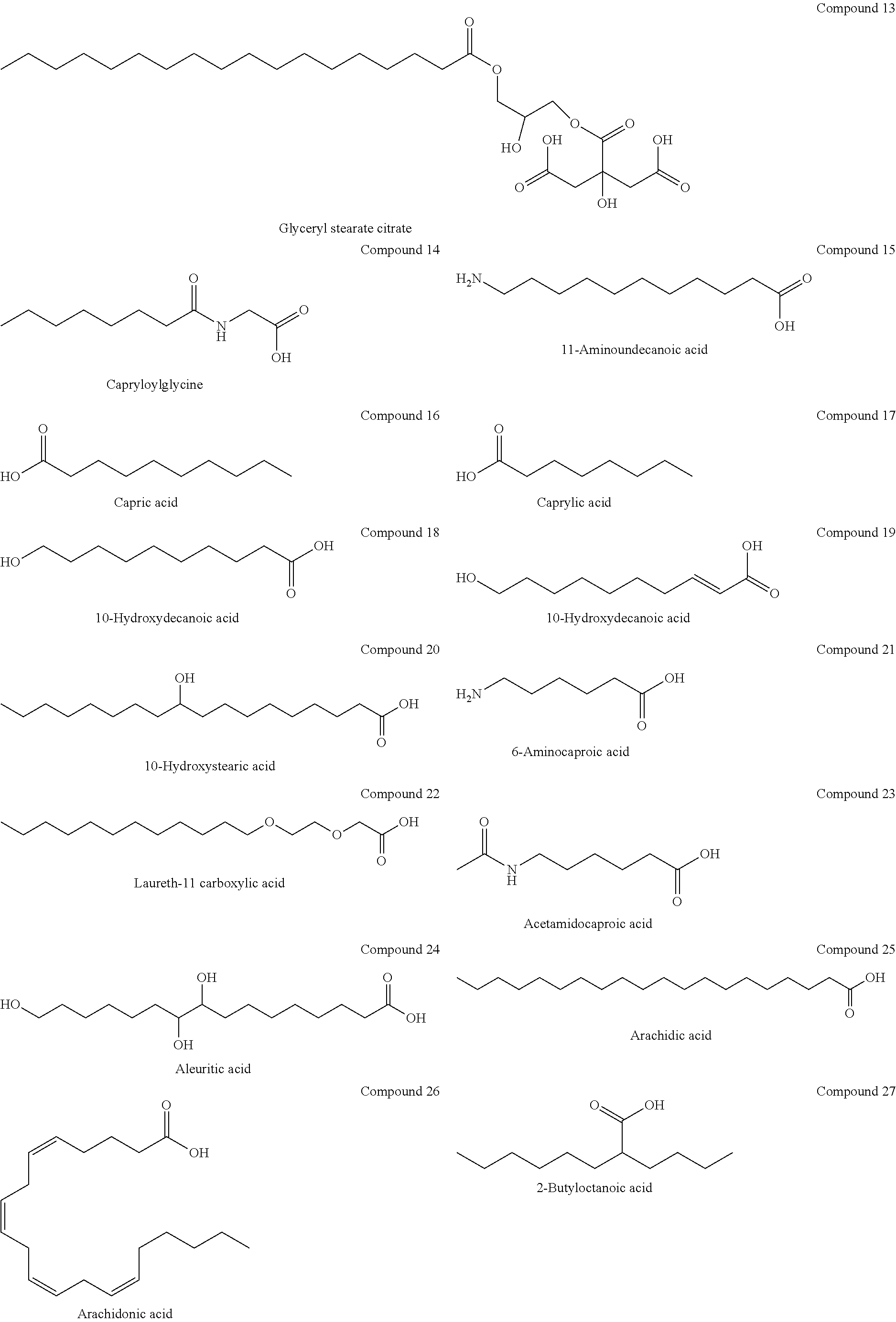

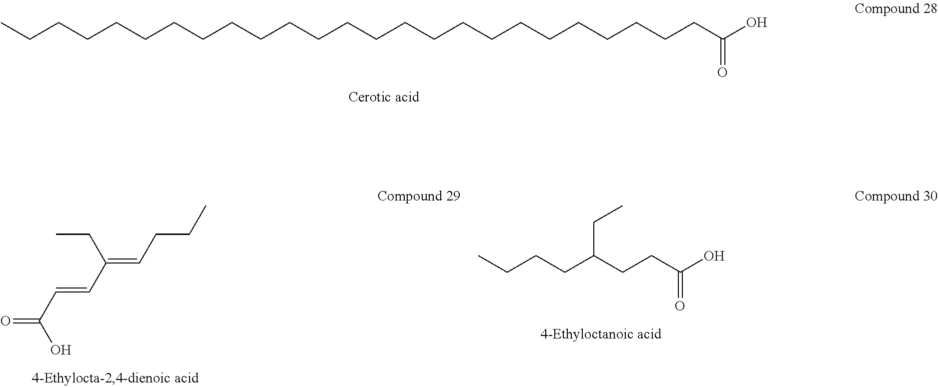 [SMILES: CCCCCCCCCCCCCCCCCCCCCCCCCC(=O)O ; CCC/C=C(/C=C/C(=O)O)CC ; CCCCC(CC)CCC(=O)O]

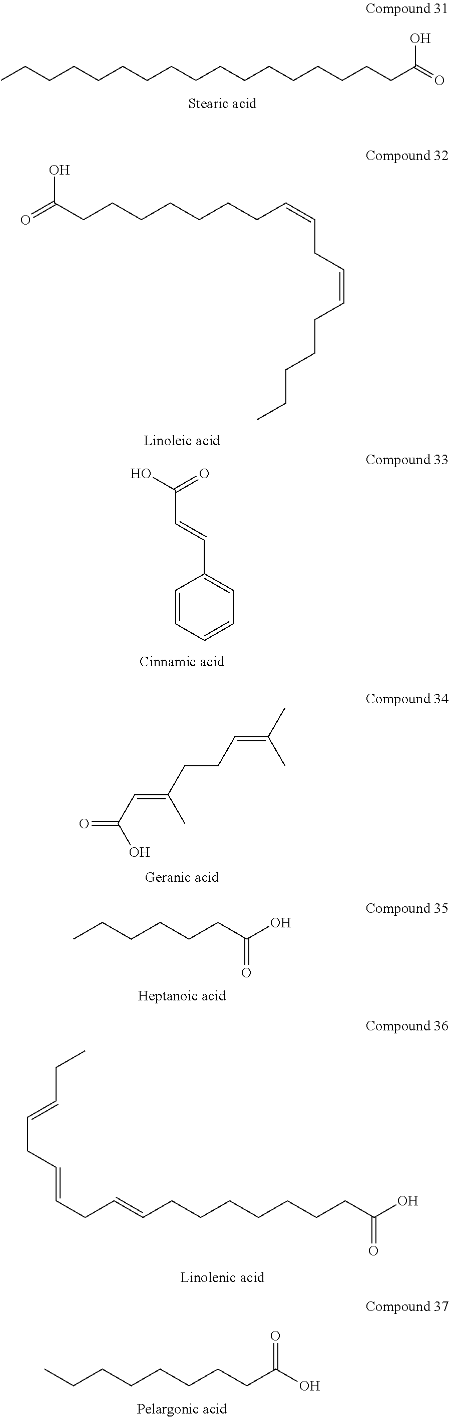

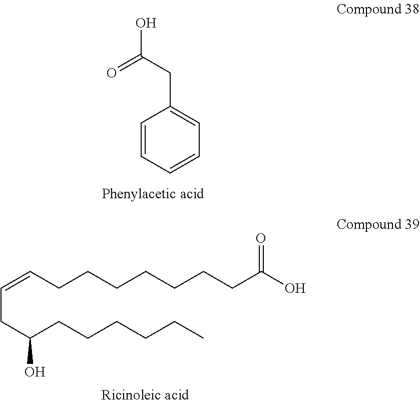 [SMILES: O=C(O)Cc1ccccc1 ; CCCCCC[C@@H](O)C/C=C\CCCCCCCC(=O)O]

According to a more preferred embodiment, the different compounds D are chosen from compounds 1 to 17, 20, 31, 32 or 36, the salts thereof, the isomers thereof, and the solvates thereof, such as hydrates, and mixtures thereof.

According to an even more preferred embodiment, the different compounds D are chosen from compounds 1, 4, 16 or 17, the salts thereof, the isomers thereof, and the solvates thereof, such as hydrates, and mixtures thereof.

According to a preferred variant, the different compounds D are chosen from compounds 1 to 39, the isomers thereof and the solvates thereof, such as hydrates, and mixtures thereof, preferably from compounds 1, 4, 16 or 17, the isomers thereof and the solvates thereof, such as hydrates, and mixtures thereof.

When one or more of the different compounds D are in salified form, they are preferably chosen from the following compounds 1′ to 11′, the isomers thereof and the solvates thereof, such as hydrates, and mixtures thereof:

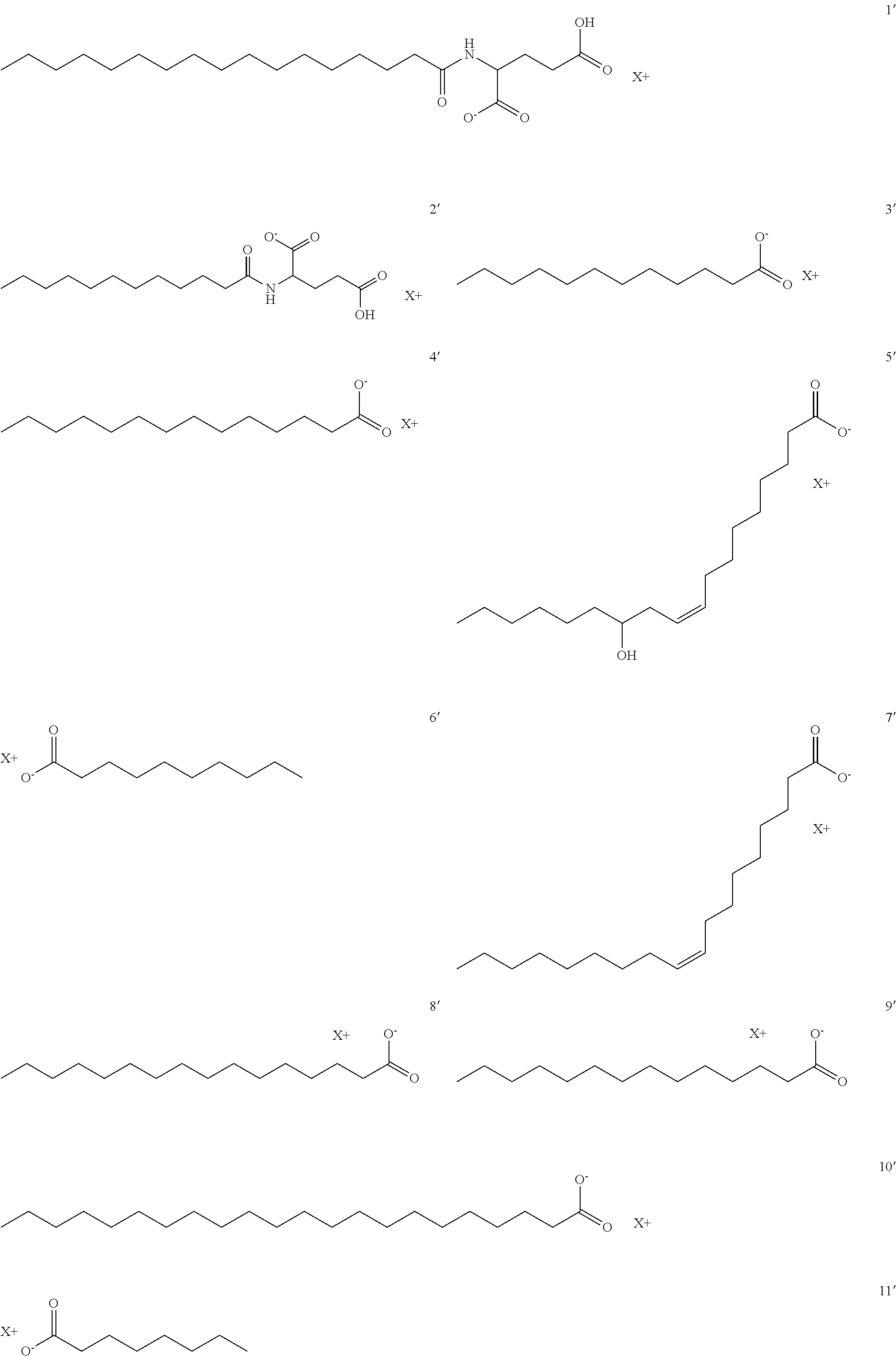

with X⁺ representing a monovalent cation, preferably a monovalent cation derived from a salt chosen from salts of alkali metals such as sodium or potassium, ammonium salts, tetra(hydroxy)(C₁-C₁₀)alkylammonium salts, di(hydroxy)(C₁-C₁₀)alkylammonium salts, tri(hydroxy)(C₁-C₁₀)alkylammonium salts, and mixtures thereof, more preferentially a monovalent cation derived from a salt chosen from salts of alkali metals such as sodium or potassium, tetra(hydroxy)(C₁-C₁₀)alkylammonium salts, and mixtures thereof.

When one or more of the different compounds D are in salified form, they are more preferentially chosen from compounds 1′ to 11′, the isomers thereof and the solvates thereof, such as hydrates, and mixtures thereof, with X⁺ representing a monovalent cation derived from a salt chosen from salts of alkali metals such as sodium or potassium, ammonium salts, tetra(C₁-C₁₀)alkylammonium salts, di(C₁-C₁₀)alkylammonium salts, tri(C₁-C₁₀)alkylammonium salts, and mixtures thereof, preferably chosen from salts of alkali metals such as sodium or potassium, tetra(C₁-C₁₀)alkylammonium salts, and mixtures thereof.

When one or more of the different compounds D are in salified form, they are even more preferentially chosen from compounds 1′ to 11′, the isomers thereof and the solvates thereof, such as hydrates, and mixtures thereof, with X⁺ representing an Na⁺, K⁺, NH₄₊ or tetra(C₁-C₁₀)alkylammonium ion.

When one or more of the different compounds D are in salified form, they are more preferentially chosen from the following compounds 1″ to 13″, the isomers thereof and the solvates thereof, such as hydrates, and mixtures thereof:

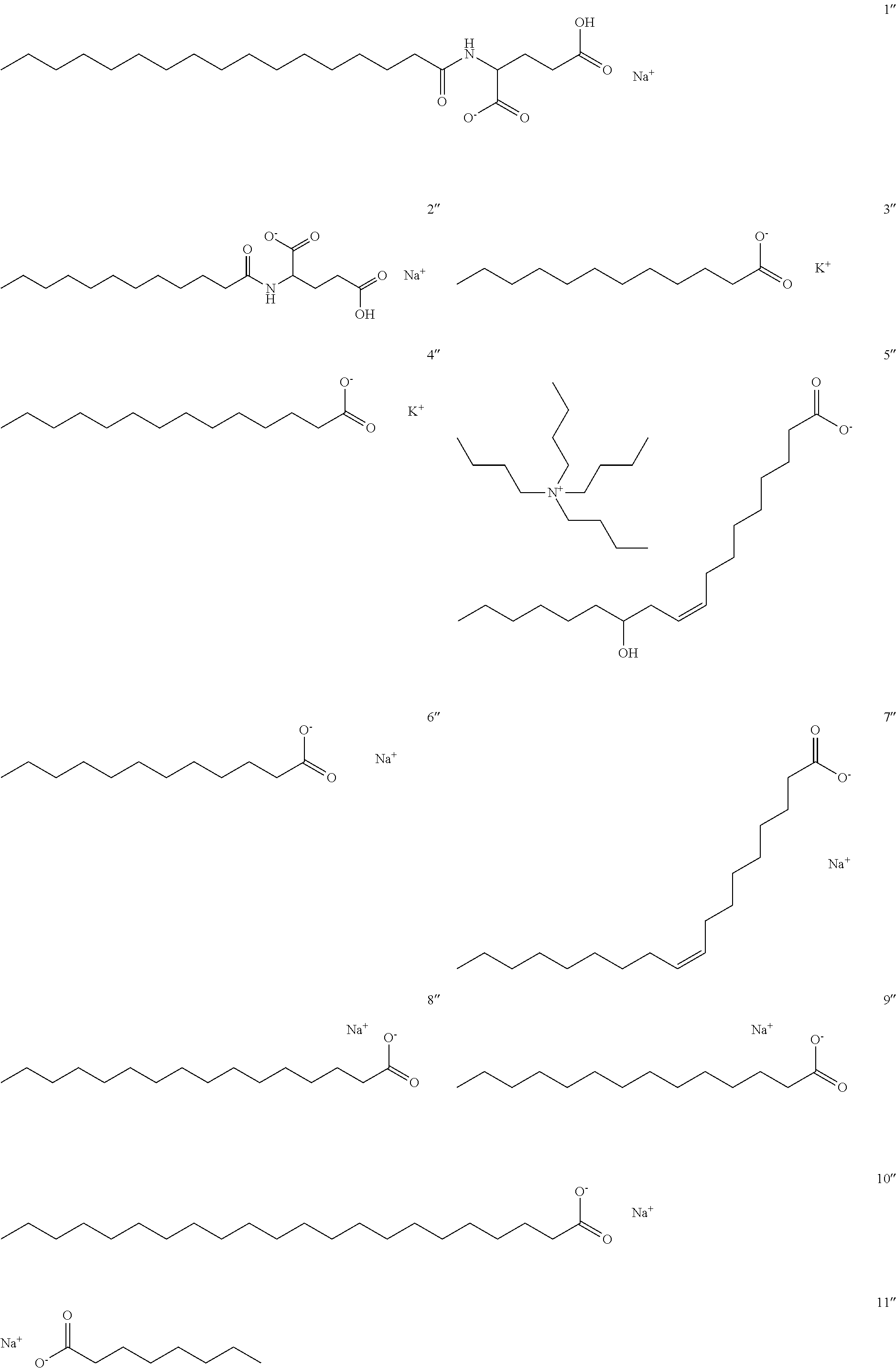

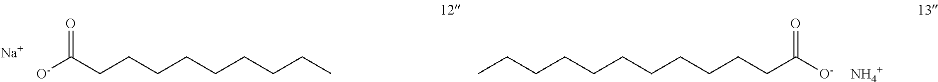

According to a particularly preferred embodiment, when one or more of the different compounds D are in salified form, they are chosen from compounds 5″, 6″, the isomers thereof and the solvates thereof, such as hydrates, and mixtures thereof.

Preferably, composition (C) comprises a total content of different compounds D of at least 0.01% by weight, preferably at least 0.05% by weight, more preferentially ranging from 0.1% to 100% by weight such as 100% by weight or 0.5% to 30% by weight, relative to the total weight of composition (C).

According to a preferred embodiment, the different compounds D are chosen from the following combinations of compounds:
the combination of caprylic acid and lauric acid, the mole ratio of the total amount of caprylic acid to the total amount of lauric acid in composition (C) preferably being equal to 3/1;
the combination of capric acid and lauric acid, the mole ratio of the total amount of capric acid to the total amount of lauric acid in composition (C) preferably being equal to 2/1;
the combination of lauric acid and oleic acid, the mole ratio of the total amount of lauric acid to the total amount of oleic acid in composition (C) preferably being from 0.25 to 0.7, more preferentially being equal to ⅓;
the combination of caprylic acid and capric acid, the mole ratio of the total amount of caprylic acid to the total amount of capric acid in composition (C) preferably being equal to 1/1;
the combination of capric acid and sodium laurate, the mole ratio of the total amount of capric acid to the total amount of sodium laurate in composition (C) preferably being equal to 4/1.

According to a more preferred embodiment, the different compounds D are chosen from the following combinations of compounds:
the combination of caprylic acid and lauric acid, the mole ratio of the total amount of caprylic acid to the total amount of lauric acid in composition (C) preferably being equal to 3/1;
the combination of capric acid and lauric acid, the mole ratio of the total amount of capric acid to the total amount of lauric acid in composition (C) preferably being equal to 2/1;
the combination of lauric acid and oleic acid, the mole ratio of the total amount of lauric acid to the total amount of oleic acid in composition (C) preferably being from 0.25 to 0.7, more preferentially being equal to ⅓;
the combination of caprylic acid and capric acid, the mole ratio of the total amount of caprylic acid to the total amount of capric acid in composition (C) preferably being equal to 1/1.

According to a most preferred embodiment, composition (C) comprises the combination of lauric acid and oleic acid, the mole ratio of the total amount of lauric acid to the total amount of oleic acid in composition (C) preferably ranging from 0.25 to 0.7, more preferentially being equal to ⅓.

When composition (C) comprises one of the combinations of two compounds described previously, the total content of the combination present in composition (C) is preferably at least 0.01% by weight, more preferentially at least 0.05% by weight, even more preferentially from 0.1% to 100% by weight, such as 100% by weight or 0.5% to 30% by weight, relative to the total weight of composition (C).

### Other Features of Composition (C)

Composition (C) is a cosmetic composition, i.e. a composition which comprises a cosmetically acceptable medium, i.e. a medium that is compatible with human keratin fibres.

Composition (C) may comprise water, preferably in a total content ranging from 0.1% to 90% by weight, relative to the total weight of the composition.

According to a preferred embodiment, composition (C) comprises a total water content of less than 30% by weight, preferably less than 10% by weight, more preferentially less than 5% by weight, even more preferentially less than 1% by weight, most preferentially less than 0.1% by weight, and better still less than 0.01% by weight relative to the total weight of composition (C), and even better still composition (C) is anhydrous.

Where appropriate, such small amounts of water may notably be introduced by ingredients of the composition that may contain residual amounts thereof.

The pH of composition (C) preferably ranges from 2 to 11, more preferentially from 3 to 9. The pH may be adjusted using an organic or mineral acid or an organic or mineral base usually used in cosmetics.

Composition (C) may comprise one or more organic solvents chosen from C₂-C₄ alcohols, ethers, polyols and polyol ethers, aromatic alcohols, hydrocarbon-based oils and mixtures thereof.

When present in composition (C), this or these organic solvents may be present in composition (C) in a total content ranging from 0.01% to 98% by weight, preferably ranging from 30% to 95% by weight relative to the total weight of composition (C).

Composition (C) is preferably liquid.

For the purposes of the present invention, the term “liquid” means, on a macroscopic scale, a product which does not have its own shape and cannot be grasped, unlike a solid. It is a fluid which adapts to the shape of the container in which it is placed at room temperature (25° C.). When poured into another container, a liquid retains its volume. A liquid thus has its own volume. At rest, the free surface of a liquid is generally flat and horizontal.

Composition (C) may be in any presentation form conventionally used for haircare application. In a non-limiting manner, composition (C) may be in the form of a lotion, serum, cream, mousse, gel, spray or lacquer.

Composition (C) may be used in a rinse-off or leave-on application.

Composition (C) may be in the form of a mask, a conditioning composition or a pre-shampoo. Composition (C) may also be in the form of a composition to be added to or mixed with a mask or a conditioning composition before application.

Composition (C) may be packaged in a pump bottle or in an aerosol container, so as to apply composition (C) in vapourized (lacquer) or foam form. In these cases, composition (C) preferably comprises at least one propellant.

According to a preferred embodiment, composition (C) comprises a total content of colouring agents of less than 0.1% by weight, preferably less than 0.01% by weight, more preferentially less than 0.01% by weight relative to the total weight of the composition; even more preferentially, the composition is free of colouring agents.

The term “colouring agent” means an oxidation dye, a direct dye or a pigment.

The term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.

The term “direct dye” means a natural and/or synthetic dye, including in the form of an extract or extracts, other than oxidation dyes. These are coloured compounds that will spread superficially on the fibre. They may be ionic or nonionic, i.e. anionic, cationic, neutral or nonionic.

According to a preferred embodiment, composition (C) comprises a total silicone content of less than 2% by weight, preferably less than 1% by weight, more preferentially less than 0.1% by weight, even more preferentially less than 0.01% by weight, most preferentially less than 0.001% by weight relative to the total weight of composition (C), and better still, composition (C) is silicone-free.

According to a preferred embodiment, composition (C) comprises a total content of cationic polymers of less than 5% by weight, preferably of less than 2% by weight, more preferentially of less than 1% by weight, even more preferentially of less than 0.1% by weight, most preferentially of less than 0.01% by weight, and better still of less than 0.001% by weight relative to the total weight of composition (C), and even better still composition (C) is free of cationic polymers.

The term “cationic polymer” means any non-silicone polymer (not comprising silicon atoms) containing cationic groups and/or groups that can be ionized into cationic groups, and not containing any anionic groups and/or groups that can be ionized into anionic groups.

According to a preferred embodiment, composition (C) comprises a total content of cationic surfactants of less than 5% by weight, preferably less than 2% by weight, more preferentially less than 1% by weight, even more preferentially less than 0.1% by weight, most preferentially less than 0.01% by weight, and better still less than 0.001% by weight relative to the total weight of composition (C), and even better still composition (C) is free of cationic surfactants.

According to a preferred embodiment, composition (C) comprises a total content of poly(oxyethylenated) compounds of less than 5% by weight, preferably of less than 2% by weight, more preferentially of less than 1% by weight, even more preferentially of less than 0.1% by weight, most preferentially of less than 0.01% by weight, and better still of less than 0.001% by weight relative to the total weight of composition (C), and even better still composition (C) is free of poly(oxyethylenated) compounds.

Examples of poly(oxyethylenated) compounds that may be mentioned include polyethylene glycols (PEGs).

Composition (C) may be applied to dry or wet keratin fibres, preferably wet keratin fibres.

The bath ratio of composition (C) applied to the keratin fibres may range from 0.02 to 10. The term “bath ratio” means the ratio between the total weight of composition (C) applied and the total weight of keratin fibres to be treated.

The process may comprise at least one additional step following step i) chosen from steps ii) to iv) below:
ii) a step of applying composition (C) to the keratin fibres, preferably for a period of at least 10 seconds, said application step optionally being performed under an airtight film of wrapper or plastic film type;
iii) a step of rinsing and/or washing the keratin fibres;
iv) a step of drying the keratin fibers in ambient air or using a heating device.

Preferably, the process comprises the additional steps ii) and iv) as described previously and performed in that order.

According to a particular embodiment, the process comprises all the additional steps ii), iii) and iv) as described previously and performed in that order.

The step of applying composition (C) may last from 10 seconds to 60 minutes, preferably from 30 seconds to 30 minutes.

The temperature of the heating device may range from 45° C. to 230° C., preferably from 45° C. to 100° C., more preferentially from 50° C. to 80° C. A hairdryer, a heating hood, an iron or a heating brush may be used, for example, as heating device.

The drying step iv) may also be a combination of drying with air or with a heating device at a temperature ranging from 50° C. to 80° C., such as a hairdryer or a heating hood, possibly followed by an ironing step, preferably with a straightening iron.

Steps i) and optionally steps ii) to iv) may be repeated as many times as required and each cycle of steps may be spaced from the next by a few minutes to a few days or weeks. Different hair treatments of the invention may be performed between different application cycles, before or after.

### Composition (C)

According to a second aspect, a subject of the present invention is a composition (C) as defined previously.

### Uses

According to a third aspect, a subject of the present invention is the use of a composition (C) as defined previously for caring for keratin fibres, preferably for giving keratin fibres a soft and/or smooth feel and/or sheen, more preferentially for giving them a soft and/or smooth feel.

According to a fourth aspect, a subject of the present invention is the use of a composition (C) as defined previously for protecting keratin fibres from moisture, preferably for limiting the formation of frizziness and/or the increase in volume of the head of hair in a humid environment.

### EXAMPLES

The following examples provide a better understanding of the invention, without, however, being limiting in nature.

The following compositions C₁ to C₄ were prepared according to the preparation protocols described hereinbelow:

TABLE 1

| | | | Mole ratio |
|---|---|---|---|
| | | | Compound |
| Compositions | Compound 1 | Compound 2 | 1/Compound 2 |
| C1 | Lauric acid | Oleic acid | 1/3 |
| C2 | Lauric acid | Caprylic acid | 1/3 |
| C3 | Lauric acid | Capric acid | 1/2 |
| C4 | Caprylic acid | Capric acid | 1/1 |

### Process for Preparing Composition C1

1 mol of lauric acid is weighed out in a flask into which 3 mol of oleic acid are added.

The mixture is heated with stirring at 70° C. for 1 hour. The temperature is allowed to return to room temperature with stirring. The product obtained is a colourless, transparent, slightly viscous, oily liquid.

### Process for Preparing Composition C2

1 mol of lauric acid is weighed out in a flask into which 3 mol of caprylic acid are added.

The mixture is heated with stirring at 70° C. for 1 hour. The temperature is allowed to return to room temperature with stirring. The product obtained is a colourless, transparent, slightly viscous, oily liquid.

### Process for Preparing Composition C3

1 mol of lauric acid is weighed out in a flask into which 2 mol of capric acid are added.

The mixture is heated with stirring at 70° C. for 1 hour. The temperature is allowed to return to room temperature with stirring. The product obtained is a colourless, transparent, slightly viscous, oily liquid.

### Process for Preparing Composition C4

1 mol of caprylic acid is weighed out in a flask into which 1 mol of capric acid is added.

The mixture is heated with stirring at 70° C. for 1 hour. The temperature is allowed to return to room temperature with stirring. The product obtained is a colourless, transparent, slightly viscous, oily liquid.

### Example 1: Evaluation of the Hairstyle Hold in a Humid Environment

The hairstyle hold, i.e. the formation of frizziness and the increase in the volume of the head of hair in a humid environment, were evaluated for compositions C1, C2 and C4 according to the evaluation protocol described hereinbelow.

### Locks Used

2.7 g locks of 27 cm-long Brazilian hair with a type IV curl were used.

### Evaluation Protocol

The locks were washed with a DOP shampoo (0.4 g/g of hair) before application of the test compositions.

81 mg of each of the test compositions were then applied to separate wet locks, wrung out beforehand and dabbed on absorbent paper. This application was made with the fingers while massaging the hair fibre.

The locks of hair were then combed, disentangled and placed in a glove box at 20% relative humidity at 25° C. for 2 hours.

The locks were then placed in an oven at 80% relative humidity at 25° C. for 24 hours.

Volume and frizziness measurements were taken by measuring the width of the lock at mid-height and compared with the value obtained for a reference lock that had not been treated with the composition according to the invention.

### Evaluation Results

The results of the measurements of the widths of the locks at mid-height in cm are summarized in the table hereinbelow:

TABLE 2

| | Width of locks at | Final width of locks |
|---|---|---|
| | mid-height (before | at mid-height |
| Compositions | application of the | (on leaving the |
| tested | compositions) | oven) |
| C1 | 2 | 3.0 |
| C2 | 2 | 3.2 |
| C4 | 2 | 3.8 |
| Reference | 2 | 4.8 |
| (Untreated lock) | | |

It can be seen that the width values of the locks at mid-height are lower for the locks treated via the process according to the invention than for the reference lock.

The process according to the invention thus makes it possible to limit the formation of frizziness and the increase in hair volume in a humid environment, even in the absence of silicone-based starting materials.

### Example 2: Evaluation of the “Smooth Feel” Via the Friction Test

The surface of natural hair is perceived as smooth, i.e. with a low coefficient of friction, whereas the surface of damaged hair is perceived as rough, i.e. with a high coefficient of friction.

Hair treatments that damage the hair, such as dyeing and bleaching, are known to increase the hair's coefficient of friction.

Consequently, the lower the coefficient of friction of the hair, the better the sensation of repaired hair and the ease with which the hair can be combed and disentangled under both dry and wet conditions.

The “smooth feel” was evaluated for composition C1 according to the evaluation protocol described hereinbelow.

### Locks Used

For the study, 3.5 cm-long locks of bleached Caucasian hair (alkaline solubility AS22) were used.

### Evaluation Protocol

In this test, the 3.5 cm-long lock is fixed in the specimen holder of a Zwickiline Z2.5 mechanical testing machine (Zwick, Germany) equipped with a 100 Newton (N) measuring cell. A normal force of 0.5 N is applied to the lock using forceps covered with disposable plastic foam. The foam is replaced after each measurement. The test machine applies a movement at a constant speed of 100 mm/min to extract the lock from the forceps. During removal, the force required to extract the hair is measured as a function of the displacement. The force/displacement curve obtained is characterized by a plateau at the end of the stroke, and the average force at the plateau is recorded. The impact of the hair treatments on the friction properties is determined by comparing the average force at the plateau for reference locks with the average force at the plateau for treated locks.

For measurements in the dry state (dry friction force), the hair is conditioned at 25° C. and 45% relative humidity.

For wet measurements (wet friction force), the hair locks are immersed in demineralized water for one minute before measurement.

Composition C1 was applied according to the leave-on protocol described hereinbelow, then dry and wet measurements were taken according to the protocol described previously.

A force ratio between treated and untreated hair was thus determined:

$${F}_{treated} ( N ) / {F}_{untreated} ( N ) .$$

### Leave-on Protocol

1) Washing of the lock with a DOP shampoo using a bath ratio of 0.4 g/g of hair; 2) Combing the lock;
3) Application of the compositions to the hair while still wet, in a bath ratio of 0.15 g/g of hair, no leave-on time;
4) Leaving the lock to dry at room temperature (22° C.).

### Results

The results of the measurements are summarized in the table below:

TABLE 3

| Composition | | F_{treated}/F_{untreated} | F_{treated}/F_{untreated} |
|---|---|---|---|
| tested | Leave-on protocol | on dry hair | on wet hair |
| C1 | Leave-on | 0.45 | 0.35 |

Lower friction forces are observed on dry and wet hair for the hair treated via the process according to the present invention, compared to the untreated hair. The hair surface is smoother, even in the absence of silicone-based starting materials. The treated hair is softer and easier to comb and to disentangle, compared to the untreated hair.

### Example 3: Evaluation of the Hairstyle Hold in a Humid Environment

The following compositions C5 to C8 were prepared. All the amounts are shown as mass percentages relative to the total weight of the composition.

TABLE 4

| | C5 | | C7 | C8 |
|---|---|---|---|---|
| | (Compar- | C6 | (Compar- | (Compar- |
| Ingrédients | ative) | (Invention) | ative) | ative) |
| Composition | — | 10 | — | — |
| C1 | | | | |
| Oleic acid | — | — | 10 | — |
| Lauric acid | — | — | — | 10 |
| Dicaprylyl | qs 100 | qs 100 | qs 100 | qs 100 |
| Ether | | | | |

The hairstyle hold, i.e. the formation of frizziness and the increase in the volume of the head of hair in a humid environment, were evaluated for compositions C5 to C8 according to the evaluation protocol described hereinbelow.

### Locks Used

2.7 g locks of 27 cm-long Brazilian hair with a type IV curl were used.

### Evaluation Protocol

The locks were washed with a DOP shampoo (0.4 g/g of hair) before application of the test compositions.

81 mg of each of the test compositions were then applied to separate wet locks, wrung out beforehand and dabbed on absorbent paper. This application was made with the fingers while massaging the hair fibre.

The locks of hair were then combed, disentangled and placed in a glove box at 20% relative humidity at 25° C. for 2 hours.

The locks were then placed in an oven at 80% relative humidity at 25° C. for 24 hours.

Volume and frizziness measurements were taken by measuring the width of the lock at mid-height and compared with the value obtained for a reference lock that had not been treated with the composition according to the invention.

### Evaluation Results

The results of the measurements of the widths of the locks at mid-height in cm are summarized in the table hereinbelow:

TABLE 5

| | Width of locks at | Final width of locks |
|---|---|---|
| | mid-height (before | at mid-height |
| Compositions | application of the | (on leaving the |
| tested | compositions) | oven) |
| C5 | 2 | 3.8 |
| (Comparative) | | |
| C6 | 2 | 2.9 |
| (Invention) | | |
| C7 | 2 | 3.4 |
| (Comparative) | | |
| C8 | 2 | 3.7 |
| (Comparative) | | |
| Reference | 2 | 5.8 |
| (Untreated lock) | | |

It can be seen that the width values of the locks at mid-height are lower for the lock treated via the process according to the invention than for the comparative and reference locks.

The process according to the invention thus makes it possible to limit the formation of frizziness and the increase in hair volume in a humid environment, even in the absence of silicone-based starting materials.

1. A process for treating keratin fibres, comprising step i) of applying to the keratin fibres a composition (C) comprising at least two different compounds D chosen independently from the compounds of formula (I) below: 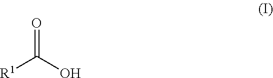
in which formula (I):
R¹ represents:
a linear or branched, saturated or unsaturated hydrocarbon-based group comprising from 6 to 26 carbon atoms, the hydrocarbon-based group being optionally substituted with one or more identical or different groups chosen from: hydroxyl (—OH), amino (—NH₂), carboxyl (—COOH), (hetero)cyclic and optionally interrupted with one or more heteroatoms or groups chosen from —O—, —CO—, —NR^{a}—, or combinations thereof; or
a (hetero)cyclic group, optionally substituted with one or more identical or different groups chosen from: hydroxyl (—OH) or R^{b}—(CO)—, wherein
R^{a} represents a hydrogen atom or a linear or branched, saturated or unsaturated hydrocarbon-based group comprising from 1 to 10 carbon atoms optionally substituted with one or more identical or different groups chosen from (—OH), amino (—NH₂) or carboxyl (—COOH);
R^{b} represents a (C₂-C₁₀)alkyl group, wherein
the salts of the compounds of formula (I) are chosen from monovalent salts of the compounds of formula (I), chosen from alkali metal salts chosen from sodium salts, potassium salts, ammonium salts, tetra(hydroxy)(C₁-C₁₀) alkylammonium salts, di(hydroxy)(C₁-C₁₀)alkylammonium salts, tri(hydroxy)(C₁-C₁₀)alkylammonium salts, or mixtures thereof; and
composition (C) comprises a total silicone content of less than 5% by weight relative to the total weight of composition (C).

2. The process according to claim 1, in which R¹ represents a linear or branched, saturated or unsaturated hydrocarbon-based group comprising from 6 to 26 carbon atoms, the hydrocarbon-based group being optionally substituted with one or more identical or different groups chosen from: hydroxyl (—OH), amino (—NH₂), carboxyl (—COOH), (hetero)cyclic and optionally interrupted with one or more heteroatoms or groups chosen from —O—, —CO—, —NR^{a}—, or combinations thereof.

3. The process according to claim 1, in which at least one of the different compounds D, are in non-salified form.

4. The process according to claim 1, in which at least two of the different compounds D are in non-salified form and the mole ratio of the total amount of one of the different compounds D in non-salified form to the total amount of another of the different compounds D in non-salified form in composition (C) ranges from 0.25 to 4.

5. The process according to claim 1, in which at least one of the different compounds D is in non-salified form and at least one of the different compounds D is in salified form and the mole ratio of the total amount of one of the different compounds D in non-salified form to the total amount of one of the different compounds D in salified form in composition (C) is greater than or equal to 1.

6. The process according to claim 1, in which composition (C) comprises not more than five different compounds D.

7. The process according to claim 1, in which the different compounds D are chosen from the following compounds 1 to 39, the salts thereof, the isomers thereof, and the solvates thereof, hydrates thereof, or mixtures thereof:

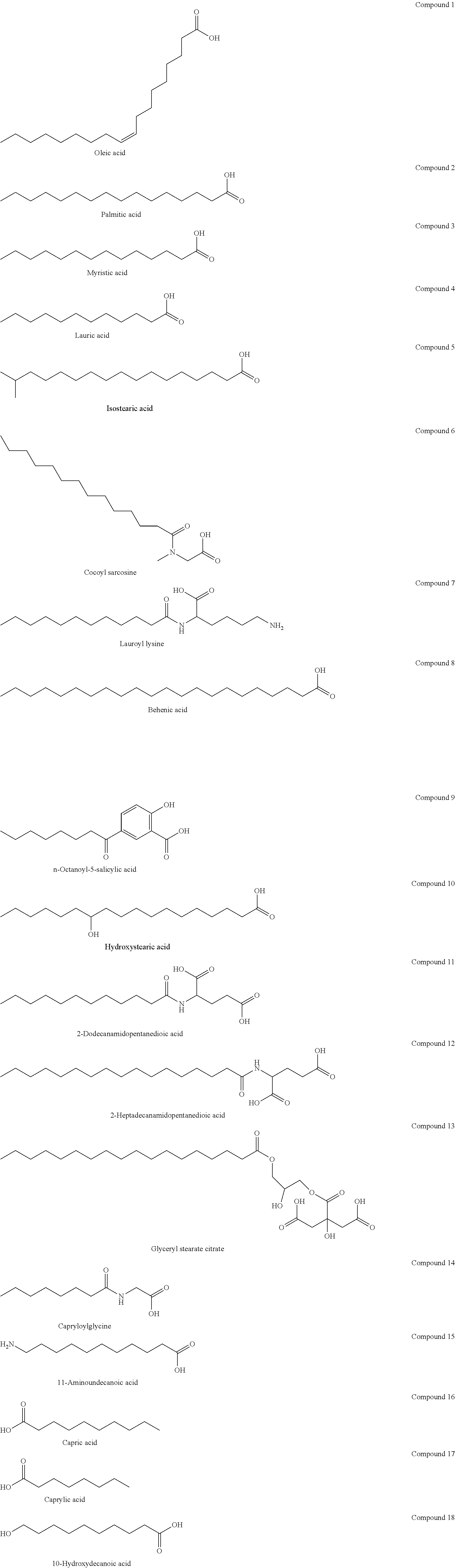

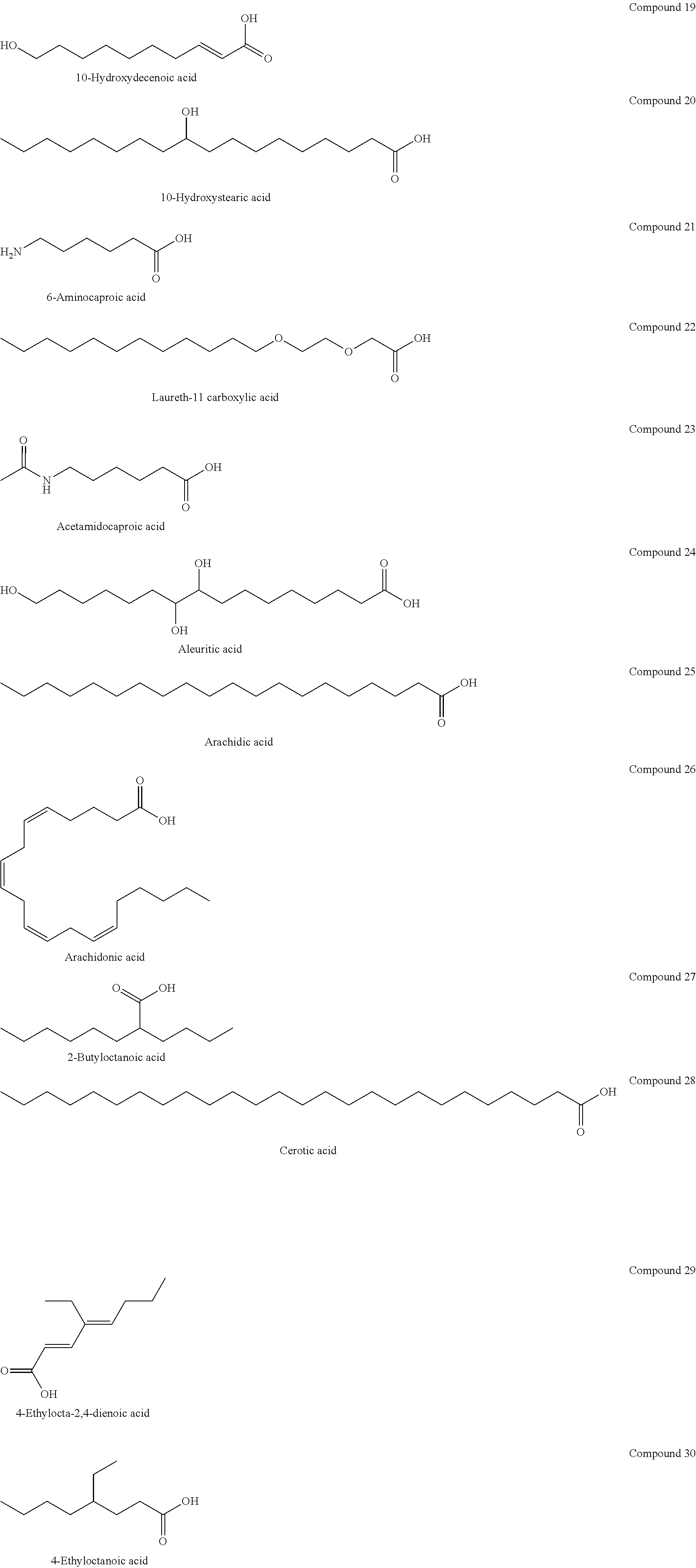

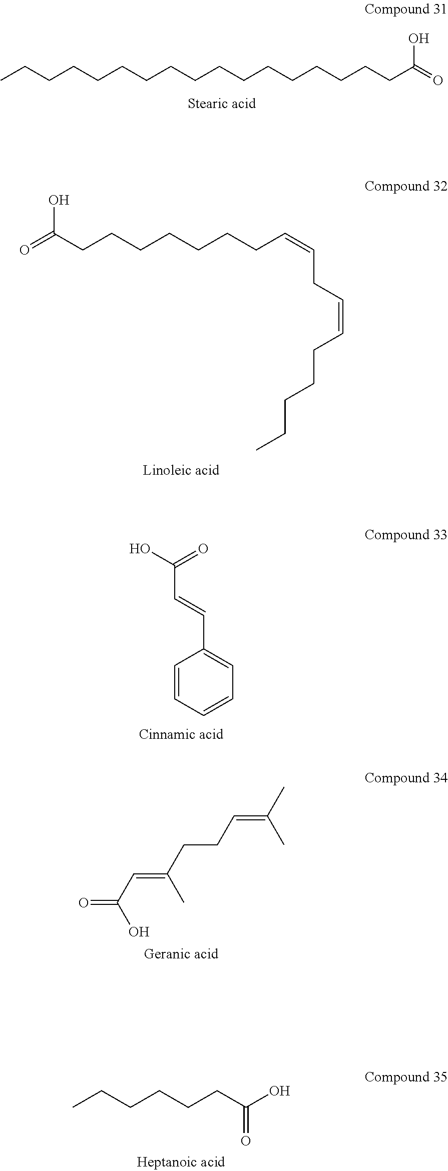

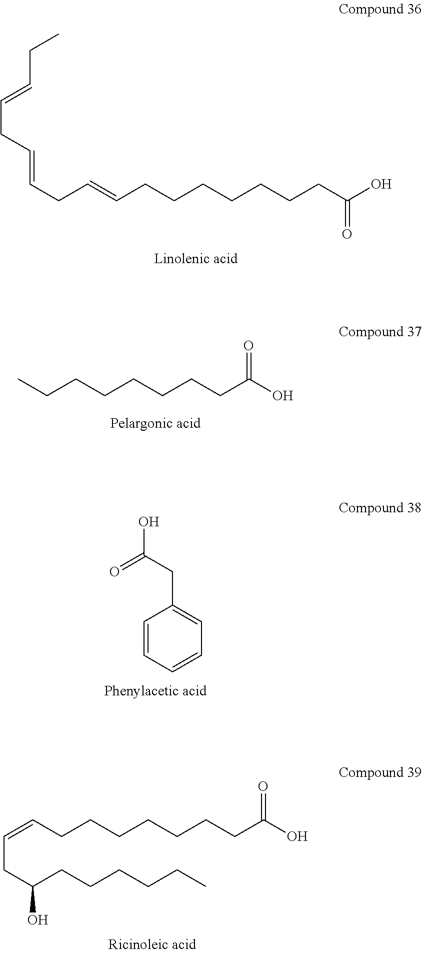 [SMILES: CC/C=C/C/C=C/C/C=C/CCCCCCCC(=O)O ; CCCCCCCCC(=O)O ; O=C(O)Cc1ccccc1 ; CCCCCC[C@@H](O)C/C=C\CCCCCCCC(=O)O]

8. The process according to claim 1, in which the different compounds D are chosen from the following combinations of compounds:
the combination of caprylic acid and lauric acid, the mole ratio of the total amount of caprylic acid to the total amount of lauric acid in composition (C) being equal to 3/1;
the combination of capric acid and lauric acid, the mole ratio of the total amount of capric acid to the total amount of lauric acid in composition (C) being equal to 2/1;
the combination of lauric acid and oleic acid, the mole ratio of the total amount of lauric acid to the total amount of oleic acid in composition (C) being from 0.25 to 0.7;
the combination of caprylic acid and capric acid, the mole ratio of the total amount of caprylic acid to the total amount of capric acid in composition (C) being equal to 1/1;
the combination of capric acid and sodium laurate, the mole ratio of the total amount of capric acid to the total amount of sodium laurate in composition (C) being equal to 4/1.

9. The process according to claim 1, in which composition (C) comprises a total content of different compounds D of at least 0.01% by weight, relative to the total weight of composition (C).

10. The process according to claim 1, in which composition (C) comprises a total silicone content of less than 2% by weight, relative to the total weight of composition (C).

11. The process according to claim 1, in which composition (C) comprises a total content of cationic polymers of less than 5% by weight relative to the total weight of composition (C).

12. The process according to claim 1, in which composition (C) comprises a total content of cationic surfactants of less than 5% by weight, relative to the total weight of composition (C).

13. The process according to claim 1, in which composition (C) comprises a total content of poly(oxyethylenated) compounds of less than 5% by weight, relative to the total weight of composition (C).

14. The process according to claim 1, in which composition (C) comprises a total water content of less than 30% by weight-relative to the total weight of composition (C).

15. The process according to claim 1, in which composition (C) comprises one or more organic solvents chosen from C₂-C₄ alcohols, ethers, polyols and polyol ethers, aromatic alcohols, hydrocarbon-based oils or mixtures thereof.

16. The process according to claim 1, in which composition (C) is applied to dry or wet keratin fibres.

17. The process according to claim 17, comprising at least one additional step successive to step i) chosen from the following steps ii) to iv): ii) a step of applying composition (C) to the keratin fibres for a period of at least 10 seconds, said application step optionally being performed under airtight conditions;
iii) a step of rinsing, washing, or rinsing and washing the keratin fibers;
iv) a step of drying the keratin fibers in ambient air or using a heating device.

18. A Composition (C), comprising:
at least two different compounds D chosen independently from the compounds of formula (I) below: 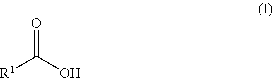
in which formula (I):
R¹ represents:
a linear or branched, saturated or unsaturated hydrocarbon-based group comprising from 6 to 26 carbon atoms, the hydrocarbon-based group being optionally substituted with one or more identical or different groups chosen from: hydroxyl (—OH), amino (—NH₂), carboxyl (—COOH), (hetero)cyclic and optionally interrupted with one or more heteroatoms or groups chosen from —O—, —CO—, —NR^{a}—, or combinations thereof; or
a (hetero)cyclic group, optionally substituted with one or more identical or different groups chosen from: hydroxyl (—OH) or R^{b}—(CO), wherein
R^{a} represents a hydrogen atom or a linear or branched, saturated or unsaturated hydrocarbon-based group comprising from 1 to 10 carbon atoms optionally substituted with one or more identical or different groups chosen from (—OH), amino (—NH₂) or carboxyl (—COOH);
R^{b} represents a (C₂-C₁₀)alkyl group, wherein
the salts of the compounds of formula (I) are chosen from monovalent salts of the compounds of formula (I), chosen from alkali metal salts chosen from sodium salts, potassium salts, ammonium salts, tetra(hydroxy)(C₁-C₁₀)alkylammonium salts, di(hydroxy)(C₁-C₁₀)alkylammonium salts, tri(hydroxy)(C₁-C₁₀)alkylammonium salts, or mixtures thereof; and
composition (C) comprises a total silicone content of less than 5% by weight relative to the total weight of composition (C).

19. A method for caring for keratin fibres, wherein the use of the composition (C) according to claim 18 confers a soft smooth feel.

20. The method according to claim 19, wherein the use of the composition (C) confers protection of the keratin fibres from moisture, thereby limiting formation of frizziness and increase in volume of the head of the keratin fibres in a humid environment.
